(19) United States
(12) Patent Application Publication
(10) Pub. No.: US 2026/0305664 A1
(43) Pub. Date: Oct. 8, 2026

(54) IMPROVEMENTS IN OR RELATING TO ORGANIC COMPOUNDS

(71) Applicant: GIVAUDAN SA, VERNIER (CH)

(72) Inventors: Adri DE KLERK, Naarden (NL); Cornelis WINKEL, Naarden (NL)

(21) Appl. No.: 19/483,526

(22) PCT Filed: May 16, 2024
(86) PCT No.: PCT/EP2024/063492

§ 371 Date: Nov. 11, 2025

(30) Foreign Application Priority Data
May 22, 2023 (GB) 2307621.9

(51) Int. Cl. A23L 27/20; A23C 19/09; A23L 23/10; C07C 233/51

(52) U.S. Cl. CPC A23L 27/204; A23C 19/0904; A23C 19/0925; A23L 23/10; C07C 233/51

(57) ABSTRACT

It is described a compound according to formula (I)

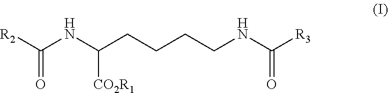

wherein either R₁ is methyl or ethyl,
and wherein R₂ and R₃ are independently selected from the group consisting of 2-phenylvinyl, phenyl, 3,4-dimethylphenyl, p-methoxyphenyl, 2-(p-methoxyphenyl)vinyl and 2-but-2-ene. Said compound can be used as an ingredient to confer, enhance, improve or modify the umami taste.

### TECHNICAL FIELD

The present invention is directed to umami flavor and methods to create it. It relates to compounds providing umami taste and savoury flavour, and to their use in compositions and consumable products.

### BACKGROUND

Umami is a flavour sensation generally associated with Asian cuisine. It has been described as savory or meaty and is characteristic of broths and cooked meats. Furthermore, improved umami taste helps make low salt products more palatable. Umami flavour has traditionally been achieved by the addition of monosodium glutamate (MSG) to foodstuffs. However, some consumers are believed to be adversely affected by glutamate salts, in particularly MSG, and consequently there remains a need for compounds that are not based on glutamate to replace or reduce reliance on such compounds for modifying the umami taste and savoury flavour of consumable products.

Amides of cinnamic acid derivatives and aromatic amines from natural sources have been reported as natural or nature-identical umami tastants in US2012308703A1, WO2013000673A1 or WO2014083202A1. Flavour compositions comprising such amides and further substances are disclosed in WO2014095564A1. A particular class of cinnamides that might generate a trigeminal effect is subject of recently published WO2019063069A1. In WO2021063942A1, putrescine bisamides and amide ester analogues with umami taste properties are disclosed. Similar structures based on ornithine as linker of the two amide functions are subject of WO2022/231908.

It has now been surprisingly found that certain bisamides based on lysine as the linker between the two amide functions have umami taste properties.

### SUMMARY

So in a first aspect of the invention, there are provided novel lysine bisamides.

In a second aspect of the invention, there is provided the use of said lysine bisamides as ingredients to confer, enhance, improve or modify the umami taste of a consumable composition.

In a third aspect of the invention, there is provided a flavour composition comprising flavour ingredients and said lysine bisamides.

In a fourth aspect of the invention, there is provided a consumable composition having umami flavour, said umami flavour being at least partially provided by the presence therein of said lysine bisamides.

There is further provided a method to confer, enhance, improve or modify the umami taste of a consumable composition by adding said lysine bisamides to the consumable composition.

The details, examples and preferences provided in relation to any particular one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### DETAILED DESCRIPTION

The present invention is based on the surprising finding that certain lysine bisamides have umami taste properties.

In a first aspect of the invention, there is provided a compound according to formula (I)

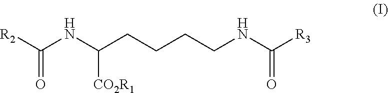

wherein R₁ is methyl or ethyl,
and wherein R₂ and R₃ are independently selected from the group consisting of 2-phenylvinyl, phenyl, 3,4-dimethylphenyl, p-methoxyphenyl, 2-(p-methoxyphenyl)vinyl and 2-but-2-ene, and wherein the compound is not methyl N²,N⁶-dibenzoyllysinate.

The compounds according to formula (I) are novel and have umami taste properties. This is surprising, as similar structures having a C5 linker between the two amide groups do not have the same organoleptic characteristics. For example, a cadaverine analogue, which is a C5 linker without any substituent, or a corresponding lysine compound with the free carboxylic group do not have umami taste. This is demonstrated by counterexamples provided below.

The compound according to formula (I) may have CC double bonds or stereocenters. It may be present in the form of any one of its stereoisomers and/or double bond isomers, or as a mixture thereof.

For example, the compound according to formula (I) is based on L-lysine as diamine compound acting as linker. L-lysine is the enantiomer naturally occurring in peptides, and it is also referred to as (S)-lysine, which means that the α-carbon is in the S configuration. Therefore, the compound according to formula (I) has also said α-carbon of the diamine linker in the S configuration. Alternatively, the compound according to formula (I) can be based on D-lysine, or on an isomeric or racemic mixture of L- and D-lysine.

For example, the compound according to formula (I) has two different substituents R₂ and R₃, independently selected from the group consisting of 2-phenylvinyl, phenyl, 3,4-dimethylphenyl, p-methoxyphenyl, 2-(p-methoxyphenyl)vinyl and 2-but-2-ene. Alternatively, the substituents R₂ and R₃ on the compound according to formula (I) are the same.

For example, the compound according to formula (I) bears two different substituents R₂ and R₃, wherein one of them is selected from the group consisting of 2-phenylvinyl, phenyl, 3,4-dimethylphenyl, p-methoxyphenyl and 2-(p-methoxyphenyl)vinyl, and the other one is 2-but-2-ene. For example, R₂ is selected from the group consisting of 2-phenylvinyl, phenyl, 3,4-dimethylphenyl, p-methoxyphenyl and 2-(p-methoxyphenyl)vinyl, and R₃ is 2-but-2-ene. Alternatively, R₂ is 2-but-2-ene and is R₃ selected from the group consisting of 2-phenylvinyl, phenyl, 3,4-dimethylphenyl, p-methoxyphenyl and 2-(p-methoxyphenyl)vinyl.

For example, the compound according to formula (I) is selected from the group consisting of methyl N⁶-cinnamoyl-N²-((E)-2-methylbut-2-enoyl)lysinate, ethyl N⁶-cinnamoyl-N²-((E)-2-methylbut-2-enoyl)lysinate, methyl N²-cinnamoyl-N⁶-((E)-2-methylbut-2-enoyl)lysinate, ethyl N²-cinnamoyl-N⁶-((E)-2-methylbut-2-enoyl)lysinate, methyl N²-((E)-3-(4-methoxyphenyl)acryloyl)-N⁶-((E)-2-methylbut-2-enoyl)lysinate, ethyl N²-((E)-3-(4-methoxyphenyl)acryloyl)-N⁶-((E)-2-methylbut-2-enoyl)lysinate, methyl N⁶-((E)-3-(4-methoxyphenyl)acryloyl)-N²-((E)-2-methylbut-2-enoyl)lysinate, ethyl N⁶-((E)-3-(4-methoxyphenyl)acryloyl)-N²-((E)-2-methylbut-2-enoyl)lysinate, methyl N²,N⁶-dicinnamoyllysinate, ethyl N²,N⁶-dicinnamoyllysinate, methyl N²,N⁶-bis((E)-2-methylbut-2-enoyl)lysinate, ethyl N²,N⁶-bis((E)-2-methylbut-2-enoyl)lysinate, methyl (E)-N⁶-benzoyl-N²-(2-methylbut-2-enoyl)lysinate, ethyl (E)-N⁶-benzoyl-N²-(2-methylbut-2-enoyl)lysinate, methyl (E)-N²-benzoyl-N⁶-(2-methylbut-2-enoyl)lysinate, ethyl (E)-N²-benzoyl-N⁶-(2-methylbut-2-enoyl)lysinate, methyl (E)-N²-(4-methoxybenzoyl)-N⁶-(2-methylbut-2-enoyl)lysinate, ethyl (E)-N²-(4-methoxybenzoyl)-N⁶-(2-methylbut-2-enoyl)lysinate, methyl (E)-N⁶-(4-methoxybenzoyl)-N²-(2-methylbut-2-enoyl)lysinate, ethyl (E)-N⁶-(4-methoxybenzoyl)-N²-(2-methylbut-2-enoyl)lysinate, methyl (E)-N⁶-(3,4-dimethylbenzoyl)-N²-(2-methylbut-2-enoyl)lysinate, ethyl (E)-N⁶-(3,4-dimethylbenzoyl)-N²-(2-methylbut-2-enoyl)lysinate, methyl (E)-N²-(3,4-dimethylbenzoyl)-N⁶-(2-methylbut-2-enoyl)lysinate, ethyl (E)-N²-(3,4-dimethylbenzoyl)-N⁶-(2-methylbut-2-enoyl)lysinate, ethyl N²,N⁶-dibenzoyllysinate, methyl N²,N⁶-bis(4-methoxybenzoyl)lysinate, ethyl N²,N⁶-bis(4-methoxybenzoyl)lysinate, methyl N²,N⁶-bis(3,4-dimethylbenzoyl)lysinate, ethyl N²,N⁶-bis(3,4-dimethylbenzoyl)lysinate, methyl N²,N⁶-bis((E)-3-(4-methoxyphenyl)acryloyl)lysinate, and ethyl N²,N⁶-bis((E)-3-(4-methoxyphenyl)acryloyl)lysinate.

In a further embodiment of the present invention, there is provided the use of the compound according to formula (I) as defined above, as an ingredient to confer, enhance, improve or modify the umami taste of a consumable composition.

For example, there is provided the use of the compound according to formula (I) which has two different substituents R₂ and R₃, independently selected from the group consisting of 2-phenylvinyl, phenyl, 3,4-dimethylphenyl, p-methoxyphenyl, 2-(p-methoxyphenyl)vinyl and 2-but-2-ene. Alternatively, there is provided the use of a compound according to formula (I) which has the same substituents R₂ and R₃.

For example, there is provided the use of the compound according to formula (I) which bears two different substituents R₂ and R₃, wherein one of them is selected from the group consisting of 2-phenylvinyl, phenyl, 3,4-dimethylphenyl, p-methoxyphenyl and 2-(p-methoxyphenyl)vinyl, and the other one is 2-but-2-ene. For example, R₂ is selected from the group consisting of 2-phenylvinyl, phenyl, 3,4-dimethylphenyl, p-methoxyphenyl and 2-(p-methoxyphenyl)vinyl, and R₃ is 2-but-2-ene. Alternatively, R₂ is 2-but-2-ene and is R₃ selected from the group consisting of 2-phenylvinyl, phenyl, 3,4-dimethylphenyl, p-methoxyphenyl and 2-(p-methoxyphenyl)vinyl.

For example, there is provided the use of the compound according to formula (I) is selected from the group consisting of methyl N⁶-cinnamoyl-N²-((E)-2-methylbut-2-enoyl)lysinate, ethyl N⁶-cinnamoyl-N²-((E)-2-methylbut-2-enoyl)lysinate, methyl N²-cinnamoyl-N⁶-((E)-2-methylbut-2-enoyl)lysinate, ethyl N²-cinnamoyl-N⁶-((E)-2-methylbut-2-enoyl)lysinate, methyl N²-((E)-3-(4-methoxyphenyl)acryloyl)-N⁶-((E)-2-methylbut-2-enoyl)lysinate, ethyl N²-((E)-3-(4-methoxyphenyl)acryloyl)-N⁶-((E)-2-methylbut-2-enoyl)lysinate, methyl N⁶-((E)-3-(4-methoxyphenyl)acryloyl)-N²-((E)-2-methylbut-2-enoyl)lysinate, ethyl N⁶-((E)-3-(4-methoxyphenyl)acryloyl)-N²-((E)-2-methylbut-2-enoyl)lysinate, methyl N²,N⁶-dicinnamoyllysinate, ethyl N²,N⁶-dicinnamoyllysinate, methyl N²,N⁶-bis((E)-2-methylbut-2-enoyl)lysinate, ethyl N²,N⁶-bis((E)-2-methylbut-2-enoyl)lysinate, methyl (E)-N⁶-benzoyl-N²-(2-methylbut-2-enoyl)lysinate, ethyl (E)-N⁶-benzoyl-N²-(2-methylbut-2-enoyl)lysinate, methyl (E)-N²-benzoyl-N⁶-(2-methylbut-2-enoyl)lysinate, ethyl (E)-N²-benzoyl-N⁶-(2-methylbut-2-enoyl)lysinate, methyl (E)-N²-(4-methoxybenzoyl)-N⁶-(2-methylbut-2-enoyl)lysinate, ethyl (E)-N²-(4-methoxybenzoyl)-N⁶-(2-methylbut-2-enoyl)lysinate, methyl (E)-N⁶-(4-methoxybenzoyl)-N²-(2-methylbut-2-enoyl)lysinate, ethyl (E)-N⁶-(4-methoxybenzoyl)-N²-(2-methylbut-2-enoyl)lysinate, methyl (E)-N⁶-(3,4-dimethylbenzoyl)-N²-(2-methylbut-2-enoyl)lysinate, ethyl (E)-N⁶-(3,4-dimethylbenzoyl)-N²-(2-methylbut-2-enoyl)lysinate, methyl (E)-N²-(3,4-dimethylbenzoyl)-N⁶-(2-methylbut-2-enoyl)lysinate, ethyl (E)-N²-(3,4-dimethylbenzoyl)-N⁶-(2-methylbut-2-enoyl)lysinate, methyl N²,N⁶-dibenzoyllysinate, ethyl N²,N⁶-dibenzoyllysinate, methyl N²,N⁶-bis(4-methoxybenzoyl)lysinate, ethyl N²,N⁶-bis(4-methoxybenzoyl)lysinate, methyl N²,N⁶-bis(3,4-dimethylbenzoyl)lysinate, ethyl N²,N⁶-bis(3,4-dimethylbenzoyl)lysinate, methyl N²,N⁶-bis((E)-3-(4-methoxyphenyl)acryloyl)lysinate, and ethyl N²,N⁶-bis((E)-3-(4-methoxyphenyl)acryloyl)lysinate.

In a further embodiment of the present invention, there is provided the use of a mixture of compounds according to formula (I) as defined above, that is at least two or more of said compounds, to confer, enhance, improve or modify the umami taste of a consumable composition.

There is further provided a flavour composition comprising at least one compound according to formula (I) as defined above, and one or more further flavour ingredients. For example, the flavor composition is an umami flavor composition.

The compound according to formula (I) may be used alone as the sole flavour component in a consumable composition or in combination with further flavour ingredients to provide a flavour composition ready for addition to a consumable composition. The further flavour ingredients may include other umami tastans and/or umami taste or savoury flavour enhancers, including MSG. The use of the compound according to formula (I) or according to formula (II) allows a considerable reduction in MSG levels, and in some cases the complete elimination of MSG.

Said other umami tastants and/or said umami taste or savoury flavour enhancers include, but are not limited to: L-Glu (glutamic acid, glutamate, for example in the form of its salts such as monosodium glutamate, monopotassium glutamate, monoammonium glutamate, calcium diglutamate, magnesium diglutamate), L-Asp (L-aspartic acid, or a salt thereof), 5′-ribonucleotides or their salts including, without limitation, calcium 5′-ribonucleotides, disodium 5′-ribonucleotides, and dipotassium 5′-ribonucleotides (e.g. inosinic acid, guanylic acid, adenosinic acid, inosinates, guanylates, and adenylates, including guanosine 5′-monophosphate, inosine 5′-monophosphate, and 5′-adenylate and their salts such as disodium guanylate, disodium inosinate, disodium adenylate; dipotassium guanylate, dipotassium inosinate, dipotassium adenylate, calcium guanylate, calcium inosinate, calcium adenylate), autolyzed or hydrolyzed proteins (e.g. autolyzed yeast, hydrolyzed yeast, hydrolyzed vegetable proteins), Koji-Aji (a nucleotide-rich yeast extract, with fermented wheat gluten and maltodextrin also containing glutamates produced by Ajinomoto Food Ingredients), and natural preparations or extracts containing one or more of the above, for example including extracts, purees or concentrates of vegetables (including mushrooms, shiitake, soy, tomato, potato, whey, kelp/seaweeds), cereals, meat, fish (e.g. shellfish, masago), milk, cheese, and egg yolks, derived from the relevant ingredient in fresh or in fermented, partially or fully hydrolyzed form (e.g. various hydrolysed proteins).

Particular examples of said other umami tastants and/or said umami taste or savoury flavour enhancers include the compounds described in UK patent application No. 0913804 and International Application No. PCT/EP2010/059916. Other non-limiting examples of umami flavour-conferring and -enhancing compounds include those described in EP 1642886, WO 2005/015158, EP 1312268, WO 2003/088768, EP 1291342 and WO 2006/003107, all of which references are incorporated herein by reference. For example, the other compounds can be selected but are not limited to N-lactoylethanolamine, (2E)-3-(1H-imidazol-4-yl)-N-[2-(1H-imidazol-4-yl)ethyl]prop-2-enamide, 2-(2,3-dihydro-1H-inden-5-yloxy)-N-(pyridin-2-ylmethyl)acetamide and 1-(2-hydroxy-4-methoxyphenyl)-3-(pyridin-2-yl)propan-1-one

Other further flavour ingredients, besides other umami tastants and/or umami taste or savoury flavour enhancers, that might be used in combination with the compound according to formula (I) or according to formula (II) to provide a flavour composition may be selected from natural flavours, artificial flavours, spices, seasonings, and the like, synthetic flavour oils and flavour aromatics and/or oils, oleoresins, essences, distillates, and extracts derived from plants, leaves, flowers, fruits, and so forth, Generally, any flavour or food additive such as those described in Chemicals Used in Food Processing, publication 1274, pages 63-258, by the National Academy of Sciences, can be used. This publication is incorporated herein by reference.

The compound according to formula (I) may be employed directly to the consumable composition or it may form a part of a flavour composition, in particular an umami flavor composition, which is subsequently admixed with the consumable composition. In a particular embodiment the compound according to formula (I) may be employed in amounts of about 0.0001 to 20%, or 0.0002-10%, more preferred 0.0005-5% by weight based on the flavor composition.

The compound according to formula (I) can additionally be used in flavour compositions in conjunction with one or more ingredients or excipients conventionally used in flavour compositions, for example carrier materials and other auxiliary agents commonly used in the art.

Suitable excipients for flavour compositions are well known in the art and include, for example, without limitation, solvents (including water, alcohol, ethanol, oils, fats, vegetable oil, and miglyol), binders, diluents, disintegranting agents, lubricants, flavour agents, coloring agents, preservatives, antioxidants, emulsifiers, stabilisers, flavour-enhancers, anti-caking agents, and the like.

Examples of such carriers or diluents for flavour compositions may be found in for example, “Perfume and Flavour Materials of Natural Origin”, S. Arctander, Ed., Elizabeth, N.J., 1960; in “Perfume and Flavour Chemicals”, S. Arctander, Ed., Vol. I & II, Allured Publishing Corporation, Carol Stream, USA, 1994; in “Flavourings”, E. Ziegler and H. Ziegler (ed.), Wiley-VCH Weinheim, 1998, and “CTFA Cosmetic Ingredient Handbook”, J. M. Nikitakis (ed.), 1st ed., The Cosmetic, Toiletry and Fragrance Association, Inc., Washington, 1988.

Other suitable and desirable ingredients of flavour compositions are described in standard texts, such as “Handbook of Industrial Chemical Additives”, ed. M. and I. Ash, 2nd Ed., (Synapse 2000).

There is further provided a consumable composition comprising at least one compound according to formula (I) or according to formula (II), or a flavour composition comprising one or more compounds according to formula (I); and a product base.

For example, the consumable composition has umami flavour, said umami flavour being at least partially provided by the presence therein of a compound according to formula (I).

The proportion of the compound according to formula (I) used in a consumable composition will depend on the nature of the use and the effect desired. For example, the proportion needed for a partial replacement of MSG will naturally be lower than that of a complete MSG replacement.

The proportion may vary between wide limits, typically between 0.01 ppm and 1000 ppm by weight of a consumable composition, more particularly between 0.1 ppm and 100 ppm, still more particularly between 1 ppm and 50 ppm or 1-5 ppm. However, these are general indications only of useful proportions, and the skilled flavourist may use proportions outside these ranges for particular effects.

By “consumable composition” is meant any composition that is taken into the mouth for ultimate spitting out or ingestion. The composition may be in any physical form, solid, liquid or gaseous. Non-limiting examples include all food products, food additives, nutraceuticals, pharmaceuticals and any product placed in the mouth including (but not limited to) chewing gum, oral care products, and oral hygiene products including but not limited to, cereal products, rice products, tapioca products, sago products, baker's products, biscuit products, pastry products, bread products, confectionery products, dessert products, gums, chewing gums, flavor or flavor-coated food/beverage containers, yeast products, baking-powder, salt and spice products, snack foods, savoury products, mustard products, vinegar products, sauces (condiments), soups, seasonings, ready-to-eat meals, gravies, nuts & nut products, processed foods, vegetable products, meat and meat products, egg products, milk and dairy products, yoghurts, cheese products, butter and butter substitute products, milk substitute products, soy products, edible oils and fat products, beverages, carbonated beverages, alcoholic drinks such as beers, wines and spirits, non-alcoholic drinks such as soft drinks or other flavoured articles, including forms requiring reconstitution including, without limitation, beverage powder, milk based beverage powder, sugar-free beverage powder, beverage syrup, beverage concentrate, coffee and tea, food extracts, plant extracts, meat extracts, condiments, gelatins, pharmaceutical and non-pharmaceutical gums, tablets, lozenges, drops, emulsions, elixirs, syrups and other preparations for making beverages, and combinations thereof.

For example, the compound according to formula (I) is suitable for consumable compositions selected from the group consisting of savoury applications, including snacks, soups, bouillon, sauces, meat/protein and ready-to-eat-meals, amongst others.

By “product base” is meant is meant the combination of all the usual art-recognised ingredients required for the particular consumable composition.

In a further aspect of the invention, there is provided a method of providing a consumable composition having umami taste, comprising the step of adding one or more compounds according to formula (I) to the consumable composition.

The compound according to formula (I) can be obtained using straightforward synthetic procedures and readily available starting materials known to person skilled in the art. Particular reaction conditions are further described in the examples.

The invention is now further described by the following non-limiting examples, which depict particular embodiments.

### Examples

### Example 1: Synthesis of ethyl N²-cinnamoyl-N⁶-((E)-2-methylbut-2-enoyl)lysinate

a) Synthesis of N⁶-(tert-butoxycarbonyl)-N²-cinnamoyllysine

Commercially available N⁶-(tert-butoxycarbonyl)lysine (5.00 g, 1 Eq, 20.3 mmol) was dissolved in a solution of sodium hydroxide (2.44 g, 3 Eq, 60.9 mmol) in water (50 ml). Tetrahydrofuran (50.0 ml) was added. Cinnamoyl chloride (3.38 g, 1 Eq, 20.3 mmol) was added dropwise while stirring and cooling with an ice/water bath. Stirring was continued for two hours at room temperature. The reaction mixture was diluted with water, acidified with a 2 M hydrochloric acid solution and twice extracted with ethyl acetate. The extracts were combined and washed with brine, dried and evaporated. The residue was purified by flash column chromatography, eluent DCM/Methanol starting with 0% methanol. The desired product was eluted with 2% methanol. 4 g of N⁶-(tert-butoxycarbonyl)-N²-cinnamoyllysine were obtained as a white solid (50%).

b) Synthesis of ethyl cinnamoyllysinate, HCl

N⁶-(tert-butoxycarbonyl)-N²-cinnamoyllysine (0.500 g, 1 Eq, 1.33 mmol) was dissolved in 25 mL of a 2M hydrochloric acid solution in ethanol. This solution was allowed to stand during two days at RT. Volatiles were removed under reduced pressure. The solid residue was taken up in ether, filtered and dried. Yield: 200 mg (40%)

c) Synthesis of ethyl N²-cinnamoyl-N⁶-((E)-2-methylbut-2-enoyl)lysinate

Ethyl cinnamoyllysinate, HCl (0.200 g, 1 Eq, 587 μmol) was dissolved in DCM (50 mL), and triethylamine (178 mg, 3 Eq, 1.97 mmol) was added. The mixture was cooled with an ice/water bath and (E)-2-methylbut-2-enoyl chloride (83.5 mg, 1.2 Eq, 788 μmol) was added dropwise while stirring. The cooling bath was removed, and stirring was continued for three hours at RT. The mixture was diluted with DCM and washed with a diluted hydrochloric acid solution. The organic layer was separated, washed with a sodium bicarbonate solution, dried and concentrated. The residue was purified by flash column chromatography, eluent DCM/Methanol starting with 0% methanol. 0.15 g of ethyl N²-cinnamoyl-N⁶-((E)-2-methylbut-2-enoyl)lysinate was obtained as a viscous paste with a purity >95% (63%).

1H NMR (600 MHz, DMSO-d6) δ ppm 1.19 (t, J=7.06 Hz, 3H) 1.28-1.36 (m, 2H) 1.38-1.49 (m, 2H) 1.62-1.67 (m, 2H) 1.68 (br d, J=1.89 Hz, 2H). 1.71-1.76 (m, 3H) 1.71-1.76 (m, 1H) 2.08 (s, 1H) 3.03-3.13 (m, 2H) 4.06-4.14 (m, 2H) 4.28-4.33 (m, 1H) 6.27 (qd, J=6.89, 1.38 Hz, 1H) 6.72 (d, J=15.84 Hz, 1H) 7.35-7.45 (m, 1H) 7.36-7.42 (m, 1H) 7.37-7.45 (m, 1H) 7.41-7.46 (m, 1H) 7.57 (d, J=7.39 Hz, 2H) 7.74 (t, J=5.68 Hz, 1H) 8.47 (d, J=7.40 Hz, 1H).

### Example 2: Synthesis of methyl N²-cinnamoyl-N⁶-((E)-2-methylbut-2-enoyl)lysinate

a) Synthesis of methyl cinnamoyllysinate, HCl

N⁶-(tert-butoxycarbonyl)-N²-cinnamoyllysine (obtained from Example 1a), 0.500 g, 1 Eq, 1.33 mmol) was dissolved in 25 mL of a 2M hydrochloric acid solution in methanol. This solution was allowed to stand over during two days at RT. Volatiles were removed under reduced pressure. The solid residue was taken up in ether, filtered and dried. Yield: 200 mg (40%)

b) Synthesis of methyl N²-cinnamoyl-N⁶-((E)-2-methylbut-2-enoyl)lysinate

Methyl cinnamoyllysinate, HCl (0.200 g, 1 Eq, 587 μmol) was dissolved in DCM (50 mL) and triethylamine (178 mg, 3 Eq, 1.97 mmol) was added. The mixture was cooled with an ice/water bath and (E)-2-methylbut-2-enoyl chloride (83.5 mg, 1.2 Eq, 788 μmol) was added dropwise while stirring. The cooling bath was removed, and stirring was continued for three hours at RT. The mixture was diluted with DCM and washed with a diluted hydrochloric acid solution. The organic layer was separated, washed with a sodium bicarbonate solution, dried and concentrated. The residue was purified by flash column chromatography. Eluent DCM/Methanol starting with 0% methanol. 0.15 g of methyl N²-cinnamoyl-N⁶-((E)-2-methylbut-2-enoyl)lysinate was obtained as a viscous paste with a purity >95% (63%).

1H NMR (600 MHz, DMSO-d6) δ ppm 1.06-1.11 (m, 1H) 1.28-1.36 (m, 2H) 1.38-1.47 (m, 2H) 1.66 (br d, J=6.89 Hz, 3H) 1.70-1.77 (m, 2H) 1.70-1.77 (m, 2H) 2.06-2.11 (m, 1H) 3.08 (q, J=6.71 Hz, 2H) 3.64 (s, 3H) 4.29-4.39 (m, 1H) 6.23-6.32 (m, 1H) 6.72 (d, J=15.84 Hz, 1H) 7.36-7.41 (m, 1H) 7.37-7.46 (m, 3H) 7.57 (br d, J=7.23 Hz, 2H) 7.68-7.81 (m, 1H) 8.49 (br d, J=7.40 Hz, 1H).

### Example 3: Synthesis of (E)-N-(5-cinnamamidopentyl)-2-methylbut-2-enamide (comparative Example

a) Synthesis of tert-butyl (5-cinnamamidopentyl)carbamate

tert-butyl (5-aminopentyl)carbamate (5 g, 24.72 mmol) was dissolved in Dichloromethane (50 ml). TEA (6.89 ml, 49.4 mmol) was added. Cinnamoyl chloride (4.12 g, 24.72 mmol) in DCM was added dropwise while stirring. Stirring was continued for 2 hours. Reaction mixture was diluted with DCM, washed with a diluted hydrochloric acid solution followed by a potassiumcar-bonate solution. Organic layer was separated, dried and concentrated to yield 7 g of the desired product as a white solid. Purity=95%.

b) Synthesis of (E)-N-(5-cinnamamidopentyl)-2-methylbut-2-enamide

tert-butyl (5-cinnamamidopentyl)carbamate (4 g, 12.03 mmol) was dissolved in Dichloromethane (100 ml). TFA (9.27 ml, 120 mmol) was added while stirring at room temperature. Solution was allowed to stand over the night. Volatiles were removed at reduced pressure (20 mbar) till a bottom temperature of 50° C. DCM (25 mL) and TEA (10.06 ml, 72.2 mmol) were added to the residue while stirring at room temperature. A solution of (E)-2-methylbut-2-enoyl chloride (1.997 g, 16.84 mmol) in 10 mL DCM was added dropwise. Stirring was continued for two hours and the homogeneous solution was allowed to stand over night. Next day solution was diluted with DCM and washed with a diluted hydrochloric acid solution followed by a potassium carbonate solution. Organic layer was separated, dried and evaporated. The residue was purified by flash column chromatography. Eluent DCM/methanol. Yield 2.5 g of the desired product as a white solid. Purity 98%. 1H NMR (600 MHz, DMSO-d6) δ ppm 1.21-1.33 (m, 2H) 1.27 (s, 1H) 1.38-1.52 (m, 4H) 1.44 (s, 1H) 1.61-1.75 (m, 6H) 1.66 (s, 1H) 1.72 (s, 1H) 2.99-3.23 (m, 4H) 3.09 (s, 1H) 3.16 (s, 1H) 6.27 (s, 1H) 6.49-6.62 (m, 1H) 6.62-6.65 (m, 1H) 7.35-7.44 (m, 4H) 7.55 (d, J=6.89 Hz, 2H) 7.72 (s, 1H) 8.04-8.10 (m, 1H) 8.10-8.13 (m, 1H).

### Example 4: Taste Evaluation—Impact of the Substituent on the C5 Linker

The following compounds were evaluated in 0.5% salt water by a panel of experienced tasters (5 male, 3 female, aged between 38 and 62).
a) 5 ppm of methyl N²-cinnamoyl-N⁶-((E)-2-methylbut-2-enoyl)-L-lysinate (compound of Example 2);
b) 10 ppm of ethyl N²-cinnamoyl-N⁶-((E)-2-methylbut-2-enoyl)-L-lysinate (compound of Example 1);
c) 30 ppm of N²-cinnamoyl-N⁶-((E)-2-methylbut-2-enoyl)-L-lysine;
d) 10 ppm of N′-cinnamoyl-N⁵-((E)-2-methylbut-2-enoyl)-L-cadaverine (compound of Example 3).

Samples a) and b) had a slightly lingering clean umami taste, while samples c) and d) did not have any umami taste.

It is demonstrated that lysine ester bisamides have an umami effect, while the analogues with an unsubstituted C5 linker, or with lysine as linker with the free carboxylic group do not show any umami effect.

### Example 5: Taste Evaluation—Comparison with C4 Linker

The following compounds were evaluated at 1 and 5 ppm by a panel of experienced tasters (5 male, 3 female, aged between 38 and 62).
a) methyl N²-cinnamoyl-N⁶-((E)-2-methylbut-2-enoyl)-L-lysinate (compound of Example 2);
b) ethyl N²-cinnamoyl-N⁶-((E)-2-methylbut-2-enoyl)-L-lysinate (compound of Example 1;
c) ethyl N²-cinnamoyl-N⁵-((E)-2-methylbut-2-enoyl)-L-ornithine; and
d) (E)-N-(4-cinnamamidobutyl)-2-methylbut-2-enamide.

Sample d) was the weakest umami compound but its umami taste profile at 5 ppm was preferred over the three other samples a)-c).

Sample a) was the strongest of the three amino acid derivatives (samples a)-c)), followed by sample b) and then sample c). The taste profile of the lysine esters derivatives (samples a) and b)) were preferred over the ornithine ester derivative (sample c). All 3 amino acid derivatives (samples a)-c)) had a slightly bitter off-note.

### Example 6: Effect on Cheese Sauce

(E)-N-(4-cinnamamidobutyl-2-methylbut-2-enamide, ethyl N²-cinnamoyl-N⁶-((E)-2-methylbut-2-enoyl)lysinate (compound of Example 1) and methyl N²-cinnamoyl-N⁶-((E)-2-methylbut-2-enoyl)lysinate (compound of Example 2) were dosed at 1 (test I) and 5 ppm (test II) in commercially available Maggi cheese sauce. An experienced panel of tasters (composed of 5 people, 3 men, 2 women aged 28-62) gave the following comments:
Test I: The compounds according to formula (I) were stronger umami than the (E)-N-(4-cinnamamidobutyl-2-methylbut-2-enamide. Both compounds according to formula (I) were considered strong umami and had a delayed umami effect. The panel agreed the methyl ester (compound of Example 2) was the stronger tastant of the two.
Test II: The compounds according to formula (I) were stronger umami than the (E)-N-(4-cinnamamidobutyl-2-methylbut-2-enamide. Both compounds according to formula (I) were considered almost too strong umami, artificial umami and had a delayed, lingering umami effect and some bitterness. The panel agreed the methyl ester (compound of Example 2) was the stronger tastant of the two.

### Example 7: Effect on Beef Bouillon

(E)-N-(4-cinnamamidobutyl-2-methylbut-2-enamide, ethyl N²-cinnamoyl-N⁶-((E)-2-methylbut-2-enoyl)lysinate (compound of Example 1) and methyl N²-cinnamoyl-N⁶-((E)-2-methylbut-2-enoyl)lysinate (compound of Example 2) were dosed at 1 ppm (test 1) and 5 ppm (test II) in commercially available Albert Heijn beef bouillon. An experienced panel of tasters (composed of 5 people, 3 men, 2 women aged 28-62) gave the following comments:
Test I: The compounds according to formula (I) were stronger umami than the (E)-N-(4-cinnamamidobutyl-2-methylbut-2-enamide. Both compounds according to formula (I) were considered strong umami and had a delayed umami effect and a long lingering. The panel agreed the methyl ester (compound of Example 2) was the stronger tastant of the two.
Test II: The compounds according to formula (I) were stronger umami than the (E)-N-(4-cinnamamidobutyl-2-methylbut-2-enamide. Both compounds according to formula (I) were considered very strong umami, artificial umami and had a delayed, lingering umami effect and tasted slightly bitter. The panel agreed the methyl ester (compound of Example 2) was the stronger tastant of the two.

1. A compound according to formula (I) 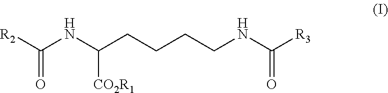
wherein either R₁ is methyl or ethyl,
and wherein R₂ and R₃ are independently selected from the group consisting of 2-phenylvinyl, phenyl, 3,4-dimethylphenyl, p-methoxyphenyl, 2-(p-methoxyphenyl)vinyl and 2-but-2-ene,
and wherein the compound is not methyl N²,N⁶-dibenzoyllysinate.

2. The compound according to claim 1, wherein one of the substituents R₂ and R₃ is selected from the group consisting of 2-phenylvinyl, phenyl, 3,4-dimethylphenyl, p-methoxyphenyl and 2-(p-methoxyphenyl)vinyl, and the other substituent is 2-but-2-ene.

3. A method of utilizing a compound according to formula (I) 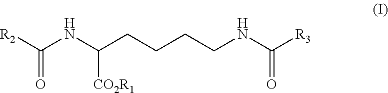
wherein either R₁ is methyl or ethyl,
and wherein R₂ and R₃ are independently selected from the group consisting of 2-phenylvinyl, phenyl, 3,4-dimethylphenyl, p-methoxyphenyl, 2-(p-methoxyphenyl)vinyl and 2-but-2-ene,
as an ingredient to confer, enhance, improve or modify the umami taste of a consumable composition, wherein the method comprises adding at least one compound according to formula (I) to a product base.

4. The method according to claim 3, wherein in the at least one compound according to formula (I) one of the substituents R₂ and R₃ is selected from the group consisting of 2-phenylvinyl, phenyl, 3,4-dimethylphenyl, p-methoxyphenyl and 2-(p-methoxyphenyl)vinyl, and the other substituent is 2-but-2-ene.

5. A flavour composition comprising
(i) at least one compound according to formula (I) as defined in claim 1, and
(ii) one or more further flavour ingredients.

6. The flavour composition according to claim 5, wherein the at least one compound according to formula (I) is present in an amount of about 0.001 to 100% by weight based on the flavor composition.

7. The flavour composition according to claim 5, wherein in the at least one compound according to formula (I) one of the substituents R₂ and R₃ is selected from the group consisting of 2-phenylvinyl, phenyl, 3,4-dimethylphenyl, p-methoxyphenyl and 2-(p-methoxyphenyl)vinyl, and the other substituent is 2-but-2-ene.

8. A consumable composition comprising:
(i) at least one compound according to formula (I), as defined in claim 1; and
(ii) a product base.

9. The consumable composition according to claim 8 selected from the group consisting of foodstuff and beverages.

10. The consumable composition according to claim 8, wherein the at least one compound according to formula (I) is present in an amount of between 0.01 ppm and 10000 ppm by weight of a consumable composition.

11. A method of providing a consumable composition having umami taste, comprising the step of adding one or more compounds as defined in claim 1 to a product base.

12. The flavour composition according to claim 6, wherein the at least one compound according to formula (I) is present in an amount of about 0.01 to 10% by weight based on the flavor composition.

13. The flavour composition according to claim 6, wherein the at least one compound according to formula (I) is present in an amount of about 0.5 to 5% by weight based on the flavor composition.

14. The consumable composition according to claim 10, wherein the at least one compound according to formula (I) is present in an amount of between 0.1 ppm and 1000 ppm by weight of a consumable composition.

15. The consumable composition according to claim 10, wherein the at least one compound according to formula (I) is present in an amount of between 1 ppm and 500 ppm by weight of a consumable composition.

16. The consumable composition according to claim 10, wherein the at least one compound according to formula (I) is present in an amount of between 5 ppm and 50 ppm by weight of a consumable composition.
